(19) United States
(12) Patent Application Publication
(10) Pub. No.: US 2015/0335547 A1
(43) Pub. Date: Nov. 26, 2015

(54) ORAL CARE COMPOSITION CONTAINING IONIC LIQUIDS

(71) Applicant: COLGATE-PALMOLIVE COMPANY, New York, NY (US)

(72) Inventors: Madhusudan Patel, Somerset, NJ (US); Mahmoud Hassan, Somerset, NJ (US)

(73) Assignee: Colgate-Palmolive Company, Piscataway, NJ (US)

(21) Appl. No.: 14/651,873

(22) PCT Filed: Dec. 20, 2012
(86) PCT No.: PCT/US2012/070960

§ 371 Date: Jun. 12, 2015

(51) Int. Cl. A61K 8/41; A61K 8/365; C07C 215/40; A61Q 11/00; C07C 59/255

(52) U.S. Cl. CPC A61K 8/416; A61Q 11/00; C07C 59/255; C07C 215/40; A61K 8/365

(57) ABSTRACT

An oral care composition comprising a compound, wherein the compound comprises: (a) a cation; and (b) an anion, wherein the compound has two or more of (i) an atomic polarizability of from 20 to 60, (ii) a Kier flexibility index of from 2 to 20 and (iii) a molar refractivity of from 3 to 10 is provided.

### BACKGROUND OF THE INVENTION

An ionic liquid is a class of salt comprising a cation and an anion that is in liquid at a temperature of 100° C. or less and commonly have melting points below room temperature. While not wishing to be bound by theory, ionic liquids generally have much lower symmetry than conventional salts and the charge of cation and anion is distributed over a larger volume of the molecule by resonance in ionic liquids which is thought to contribute to their liquid state at much lower temperatures than conventional salts (e.g. NaCl, mp 801° C.). Ionic liquids are often composed of a cation comprising a heterocyclic ring and a counter anion, often inorganic in nature. The nature of the cation and anion will determine the hydrophobicity, viscosity, density and other physical parameters and properties of the ionic liquid.

Ionic liquids have been evaluated as environmentally-friendly or ‘green’ alternatives to conventional organic solvents for a wide range of organic synthetic applications. Ionic liquids have unique characteristics that distinguish them from conventional organic solvents. For example, ionic liquids are non-volatile (i.e. they do not evaporate readily into the atmosphere), they have a high polarity and charge density, they may be hydrophobic or hydrophilic, and they have unique solvating properties. As such, ionic liquids are known to be used in cleaning compositions (for example, as disclosed in US 2006/0090777 A1 and U.S. Pat. No. 7,939,485 B2). A range of ionic liquids are commercially available, or they may be readily synthesized by simple ion-exchange reactions.

A biofilm is a structured group of microorganisms encapsulated within a self-developed polymeric extracellular matrix. Biofilms are typically adhered to a living or inert surface. In the human or animal body biofilms can form on any internal or external surface. Biofilms have been found to be involved in a wide variety of microbial infections in the body and cause a number of conditions including urinary tract infections, middle-ear infections, and in particular, diseases of the oral cavity.

Dental plaque is formed from a biofilm precursor, and is present to some degree on virtually all dental surfaces whether in the oral cavity or on dental instruments used by dental professionals. It comprises a dense microbial layer consisting of a mass of microorganisms embedded in a polysaccharide matrix. Plaque may form on any part of the tooth surface, and is found particularly at the gingival margin, and in cracks in the enamel. The danger associated with the formation of plaque on the teeth lies in the tendency of plaque to build up and eventually produce gingivitis, periodontitis and other types of periodontal disease, as well as dental caries and dental calculus. Dental plaque formation is also related to the feeling of a fuzzy tongue in an unclean oral cavity and as such addressing dental plaque formation can address cleaning the tongue.

Plaque itself adheres very firmly to dental surfaces and rapidly reforms on the tooth surface after it is removed. Current plaque removal methods rely primarily on the mechanical removal of plaque. These methods, which include brushing, brushing with an abrasive toothpaste, flossing, using interdental cleaners, scraping, using sonic energy (e.g. Sonicare toothbrushes) and ultrasound (e.g. Ultreo toothbrushes), in part, rely on a good brushing or flossing technique which may consumers simply do not possess. Moreover, these methods are particularly inefficient in removing stubborn plaque, or plaque hidden deep within cavities and fissures of teeth, or within gum pockets.

It is also known in the art to incorporate antimicrobial agents in oral compositions which destroy or retard the growth of bacteria. However, bacteria present in a biofilm or plaque deposit exhibit increased resistance to antimicrobial agents because the dense extracellular matrix and the outer layer of cells protect the bacteria found in the interior of the deposit from the effects of the antimicrobial agents.

There is therefore the need to provide improved methods and compositions for removing plaque which mitigate some of the inefficiencies resulting from a poor brushing/flossing technique and which effectively remove plaque hidden between teeth, within cavities and fissures of teeth, and in gum pockets.

### SUMMARY OF THE INVENTION

A first aspect of the present invention provides an oral care composition comprising a compound, wherein the compound comprises:
(a) a cation; and
(b) an anion
   wherein the compound has two or more of
(i) an atomic polarizability of from 20 to 60,
(ii) a Kier flexibility index of from 2 to 20 and
(iii) a molar refractivity of from 3 to 10.
   (For the purposes of this invention, apol=atomic polarizability, KierFlex=compound flexibility and SMR=molar refractivity)

A second aspect of the present invention provides an oral care composition comprising a compound, wherein the compound comprises:
(a) a cation comprising a positively charged nitrogen atom and
(b) an anion
   wherein the compound has two or more of
(i) an atomic polarizability of from 20 to 60,
(ii) a Kier flexibility index of from 2 to 20 and
(iii) a molar refractivity of from 3 to 10.

Optionally, the compound has an atomic polarizability of from 20 to 45. Further optionally the oral care composition has an atomic polarizability of from 22 to 40. Optionally the compound has a Kier flexibility index of from 3 to 10. Optionally the compound has a molar refractivity of from 3 to 7.

Optionally the compound has two or more of
(i) an atomic polarizability of from 28 to 40,
(ii) a Kier flexibility index of from 4 to 15 and
(iii) a molar refractivity of from 4 to 7.

Optionally the compound has
(i) an atomic polarizability of from 30 to 38 and
(ii) a Kier flexibility index of from 5 to 14 and
(iii) a molar refractivity of from 5 to 6.

Optionally the compound is selected from one or more of EMIM Ac (1-ethyl-3-methylimidazolium acetate), 1-OHEt-EMIM BF4 (1-hydroxyethyl-3-methylimidazoliumtetrafluoroborate), 1-OHEt-EMIM Cl (1-hydroxyethyl-3-methylimidazolium chloride), 1-OHEt-EMIM Br (1-hydroxyethyl-3-methylimidazolium bromide), 1-OHEt-EMIM Ac (1-hydroxyethyl-3-methylimidazolium acetate), 1-OHEt-EMIM SO4 (1-hydroxyethyl-3-methylimidazolium sulphate), 1-OHPr-EMIM Cl) 1-hydroxypropyl-3-methylimidazolium chloride), 1-OHPr-EMIM Br (1-hydroxypropyl-3-methylimidazolium bromide), 1-OHPr-EMIM Ac (1-hydroxypropyl-3-methylimidazolium acetate), 1-OHPr-EMIM SO4 (1-hydroxypropyl-3-methylimidazolium sulphate), 3(4-OH-Bu)-EMIM Cl (3-(4-hydroxybutyl)-1-methylimidazolium chloride, 3(4-OH-Bu)-EMIM Br (3-(4-hydroxybutyl)-1-methylimidazolium bromide), 3(4-OH-Bu)-EMIM Ac (3-(4-hydroxybutyl)-1-methylimidazolium acetate), 3(4-OH-Bu)-EMIM SO4 ((3-(4-hydroxybutyl)-1-methylimidazolium sulphate), 1-Me-3(2-PrOEt)-EMIM Cl, 1-Me-3(2-PrOEt)-EMIM Ac, 1-Me-3(2-PrOEt)-EMIM SO4, 1,2-diMe-4Pr-PZSO4, 1,2,4-triMe-PZCl (1,2,4-trimethyl pyrazolium chloride), 1,2,4-triMe-PZBr (1,2,4-trimethyl pyrazolium bromide), 1,2,4-triMe-PZAc (1,2,4-trimethyl pyrazolium acetate), 1,2-diMe-4-Et-PZMeSO4 (1,2-dimethyl-4-ethyl-pyrazolium methylsulphate), 1,2-diMe-4-Et-PZCl (1,2-dimethyl-4-ethyl-pyrazolium chloride), 1,2-diMe-4-Et-PZ (1,2-dimethyl4-ethyl-pyrazolium bromide), 1,2-diMe-4-Et-PZAc (1,2-dimethyl-4-ethyl-pyrazolium acetate), 1,2-diMe-4-Et-PZSO4) (1,2-dimethyl-4-ethyl-pyrazolium methylsulphate), 1,2-diMe-4-Pr-PZCl (1,2-dimethyl-4-propyl-pyrazolium chloride), 1,2-diMe-4-Pr-PZBr (1,2-dimethyl-4-propyl-pyrazolium bromide), 1,2-diMe-4-Pr-PZAc (1,2-dimethyl-4-propyl-pyrazolium acetate), 1,2-diMe-4-Bu-PZSO4 (1,2-dimethyl-4-butylpyrazolium methylsulphate), 1,2-diMe-4-Bu-PZCl (1,2-dimethyl-4-butylpyrazolium chloride), 1,2-diMe-4-Bu-PZBr ((1,2-dimethyl-4-butylpyrazolium bromide), 1,2-diMe-4-Bu-PZAc (1,2-dimethyl-4-butylpyrazolium acetate), choline sulphate, choline bromide, 2-chloro-3-hydroxypropyltrimethyl ammonium chloride, 2-chloro-3-hydroxypropyltrimethyl ammonium bromide, 2-chloro-3-hydroxypropyltrimethyl ammonium acetate, 2-chloro-3-hydroxypropyltrimethyl ammonium sulphate, 1,1-dimethyl-4-hydroxyethylpiperazinium chloride, 1,1-dimethyl-4-hydroxyethylpiperazium bromide, 1,1-dimethyl-4-hydroxyethylpiperazium acetate, 1,1-dimethyl-4-hydroxyethylpiperazinium methylsulphate, dimethyl(1,2-dihydroxypropyl)methylammonium chloride, dimethyl(1,2-dihydroxypropyl)methylammonium bromide, dimethyl(1,2-dihydroxypropyl)methylammonium acetate, dimethyl(1,2-dihydroxypropyl)methylammonium methyl sulphate, 1-hydroxy-1-cyano-2-trimethylamine-ethane chloride, 1-hydroxy-1-cyano-2-trimethylamine-ethane bromide, 1-hydroxy-1-cyano-2-trimethylamine-ethane acetate, 1-hydroxy-1-cyano-2-trimethylamine-ethane methyl sulphate, Tris(2-hydroxyethyl)methylammonium methylsulphate (Tris(2-OH-Et)MEAMeSO4), Tris(2-hydroxyethyl)methylammonium chloride (Tris(2-OH-Et)MEACl), Tris(2-hydroxyethyl)methylammonium bromide (Tris(2-OH-Et)MEABr), Tris(2-hydroxyethyl)methylammonium acetate (Tris(2-OH-Et)MEAAc), Bis(hydroxpropyl)-2-hydroxyethylmethylammonium methylsulphate (Bis(2-OH—Pr)2-OH-Et MEA MeSO4), Bis(hydroxpropyl)-2-hydroxyethylmethylammonium chloride (Bis(2-OH—Pr)2-OH-Et MEA Cl), Bis(hydroxpropyl)-2-hydroxyethylmethylammonium bromide (Bis(2-OH—Pr)2-OH-Et MEA Br), Bis(hydroxpropyl)-2-hydroxyethylmethylammonium acetate (Bis(2-OH—Pr)2-OH-Et MEA Ac), Bis(hydroxpropyl)-2-hydroxyethylmethylammonium methylsulphate (Bis(2-OH—Pr)2-OH-Et MEA MeSO4), Bis(hydroxybutyl)-2-hydroxyethylmethylammonium chloride (Bis(2-OH-Bu)2-OH-Et MEA Cl), B is (hydroxybutyl)-2-hydroxyethylmethylammonium bromide (Bis(2-OH-Bu)2-OH-Et MEA Br), Bis(hydroxybutyl)-2-hydroxyethylmethylammonium acetate (Bis(2-OH-Bu)2-OH-Et MEA Ac), Bis(2-OH-Bu)2-OH-Et MEA Ac, Bis(hydroxybutyl)-2-hydroxyethylmethylammonium methylsulphate, Bis(hydroxypentyl)-2-hydroxyethylmethylammonium chloride (Bis(2-OH-Pe)2-OH-Et MEA Cl), Bis(hydroxypentyl)-2-hydroxyethylmethylammonium bromide (B is (2-OH-Pe)2-OH-Et MEA Br), Bis(hydroxypentyl)-2-hydroxyethylmethylammonium acetate (Bis(2-OH-Pe)2-OH-Et MEA Ac), 2-(2-methylmorpholin-4-yl)ethan-1-amine-diCl, 2-(2-methylmorpholin-4-yl)ethan-1-amine-diBr, 2-(2-methylmorpholin-4-yl)ethan-1-amine-diOAC, 2-(2-methylmorpholin-4-yl)ethan-1-amine-diSO4, 1-(2-methoxyethyl)pyrrolidin-3-amine-diCl, 1-(2-methoxyethyl)pyrrolidin-3-amine-diBr, 1-(2-methoxyethyl)pyrrolidin-3-amine-diAc, 1-(2-methoxyethyl)pyrrolidin-3-amine-diSO4, 4-amino-3-[(dimethylamino)methyl]butan-2-ol-diCl, 4-amino-3-[(dimethylamino)methyl]butan-2-ol-diBr, 4-amino-3-[(dimethylamino)methyl]butan-2-ol-diAc, 4-amino-3-[(dimethylamino)methyl]butan-2-ol-diSO4, 3-(morpholin-4-yl)propan-1-amine-diCl, 3-(morpholin-4-yl)propan-1-amine-diBr, 3-(morpholin-4-yl)propan-1-amine-diAc, 3-(morpholin-4-yl)propan-1-amine-diSO4, 3-(2-methylpiperazin-1-yl)propan-1-ol-diCl, 3-(2-methylpiperazin-1-yl)propan-1-ol-diBr, 3-(2-methylpiperazin-1-yl)propan-1-ol-diAc, 3-(2-methylpiperazin-1-yl)propan-1-ol-diSO4, 1-butyl pyridinium chloride, 1-butyl pyridinium bromide, 1-butyl pyridinium acetate, 1-butyl pyridinium Mesulphate, 1-butyl-4-methyl pyridinium chloride, 1-butyl-4-methyl pyridinium bromide, 1-butyl-4-methyl pyridinium acetate, 1-butyl-4-methyl pyridinium Mesulphate, 1-butyl-3-methyl pyridinium chloride, 1-butyl-3-methyl pyridinium bromide, 1-butyl-3-methyl pyridinium acetate, 1-butyl-3-methyl pyridinium Mesulphate, EMPL Cl (1-ethylmethylpyrrolidinium chloride), EMPL Br (1-ethylmethylpyrrolidinium bromide), EMPL PF6 (1-ethylmethylpyrrolidinium hexafluorophosphate), EMPL BF4 (1-ethylmethylpyrrolidinium tetrafluoroborate), EMPL Ac (1-ethylmethylpyrrolidinium acetate), EMPL MeSO4 (1-ethylmethylpyrrolidinium methylsulphate), BMPL Cl (1-butylmethylpyrrolidinium chloride), BMPL Br (1-butylmethylpyrrolidinium bromide), BMPL Ac ((1-butylmethylpyrrolidinium acetate), BMPL MeSO4 (1-butylmethylpyrrolidinium methylsulphate), BMPL I (1-butylmethylpyrrolidinium iodide), BMPL BF4 (1-butylmethylpyrrolidinium tetrafluoroborate), BMPL PF6 (1-butylmethylpyrrolidinium hexafluorophosphate, or any combination thereof

Optionally the compound is selected from one or more of 1-propanaminium-2-chloro-3-hydroxy-N,N,N-trimethylchloride, 2,3-dihydroxypropyl(trimethyl)azanium chloride, 1-butylpyridinium bromide, 1-butyl-4-methylpyridinium chloride, 1-butyl-4-methylpyridinium bromide, 1-butyl-4-methylpyridinium iodide, 1-butyl-4-methylpyridinium tetrafluoroborate, 1-butyl-4-methylpyridinium hexafluorphosphate, 1-ethyl-1-methylpyrrolidinium hexafluorophosphate, 1-ethyl-1-methylpyrrolidinium tetrafluoroborate, 1-butyl-1-methylpyrrolidinium, 1-butyl-1-methylpyrrolidinium bromide, 1-butyl-1-methylpyrrolidinium iodide, 1-butyl-1-methylpyrrolidinium tetrafluoroborate, 1-butyl-1-methylpyrrolidinium hexafluorophosphate or any combination thereof.

Optionally the compound is choline bitartrate.

Optionally the composition is for removing or reducing plaque. Optionally the composition is for inhibiting growth of bacteria. Optionally the composition is for teeth whitening. Optionally the composition is for preventing or treating a disease or condition of the oral cavity.

According to a further aspect of the present invention there is provided a method of removing or reducing plaque; inhibiting or reducing the growth of bacteria; or whitening teeth; from the oral cavity of the a subject comprising administering a therapeutically effective amount of a composition comprising a compound, wherein the compound comprises:

(a) a cation; and
(b) an anion;
wherein the compound has two or more of

(i) an atomic polarizability of from 20 to 60,

(ii) a Kier flexibility index of from 2 to 20 and

(iii) a molar refractivity of from 3 to 10.

According to a further aspect of the present invention, in the methods of removing or reducing plaque; inhibiting or reducing the growth of bacteria; or whitening teeth, the compound comprises:

(a) a cation comprising a positively charged nitrogen atom; and
(b) an anion;
wherein the compound has two or more of

(i) an atomic polarizability of from 20 to 60,

(ii) a Kier flexibility index of from 2 to 20 and

(iii) a molar refractivity of from 3 to 10.

According to a further aspect of the present invention, in the methods of removing or reducing plaque; inhibiting or reducing the growth of bacteria; or whitening teeth, the compound comprises:

(a) a cation comprising a positively charged nitrogen atom; and
(b) an anion;
wherein the compound has two or more of

(i) an atomic polarizability of from 28 to 40;

(ii) a Kier flexibility index of from 4 to 15; and

(iii) a molar refractivity of from 4 to 7.

According to a further aspect of the invention, in the methods of removing or reducing plaque; inhibiting or reducing the growth of bacteria; or whitening teeth, the compound comprises:

(a) a cation comprising a positively charged nitrogen atom; and
(b) an anion;
wherein the compound has two or more of

(i) an atomic polarizability of from 30 to 38;

(ii) a Kier flexibility index of from 5 to 14; and

(iii) a molar refractivity of from 5 to 6.

According to a further aspect of the present invention there is provided a composition comprising a compound, wherein the compound comprises:

(a) a cation; and
(b) an anion;
wherein the compound has two or more of

(i) an atomic polarizability of from 20 to 60,

(ii) a Kier flexibility index of from 2 to 20 and

(iii) a molar refractivity of from 3 to 10

for use in the manufacture of a medicament for removing or reducing plaque, for inhibiting or reducing the growth of bacteria, for whitening teeth in the oral cavity of a subject.

According to a further aspect of the invention there is also provided a composition comprising a compound, wherein the compound comprises:

(a) a cation comprising a positively charged nitrogen atom; and
(b) an anion;
wherein the compound has two or more of

(i) an atomic polarizability of from 20 to 60,

(ii) a Kier flexibility index of from 2 to 20 and

(iii) a molar refractivity of from 3 to 10

for use in the manufacture of a medicament for removing or reducing plaque, for inhibiting or reducing the growth of bacteria, for whitening teeth in the oral cavity of a subject.

According to a further aspect of the invention there is also provided a composition comprising a compound, wherein the compound comprises:

(a) a cation comprising a positively charged nitrogen atom; and
(b) an anion;
wherein the compound has two or more of

(i) an atomic polarizability of from 28 to 40,

(ii) a Kier flexibility index of from 4 to 15 and

(iii) a molar refractivity of from 4 to 7

for use in the manufacture of a medicament for removing or reducing plaque, for inhibiting or reducing the growth of bacteria, for whitening teeth in the oral cavity of a subject.

According to a further aspect of the invention there is also provided a composition comprising a compound, wherein the compound comprises:

(a) a cation comprising a positively charged nitrogen atom; and
(b) an anion;
wherein the compound has two or more of

(i) an atomic polarizability of from 30 to 38,

(ii) a Kier flexibility index of from 5 to 14 and

(iii) a molar refractivity of from 5 to 6

for use in the manufacture of a medicament for removing or reducing plaque, for inhibiting or reducing the growth of bacteria, for whitening teeth in the oral cavity of a subject.

### DESCRIPTION OF THE INVENTION

It should be understood that the detailed description, and specific examples, while indicating embodiments of the invention, are intended for purposes of illustration only and are not intended to limit the scope of the invention.

As used throughout, ranges are used as shorthand for describing each and every value that is within the range. Any value within the range can be selected as the terminus of the range.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “about,” when applied to the value for a parameter of a composition or method of this invention, indicates that the calculation or the measurement of the value allows some slight imprecision without having a substantial effect on the chemical or physical attributes of the composition or method. If, for some reason, the imprecision provided by “about” is not otherwise understood in the art with this ordinary meaning, then “about” as used herein indicates a possible variation of up to 5% in the value.

As referred to herein, all compositional percentages are by weight of the total composition, unless otherwise specified.

The following description of the preferred embodiments is merely exemplary in nature and is in no way intended to limit the invention, its application, or uses.

Ionic liquids are a class of organic salts consisting of cation and anion components with melting points below 100° C. Ionic liquids typically contain an organic cation, such as 1-alkyl-3-methyl-imidazolium or N-alkylpyridinium, together with an anion component that could either be inorganic or organic, for example halides (chloride, bromide), or (trifluoromethyl)sulfonylamide. The physicochemical properties of ionic liquids can be tuned by varying the combinations of ions and by sidechain modifications to the cation component. Ionic liquids are considered “green solvents” due to the fact that they are not flammable, have very low vapor pressures and are recyclable.

Certain important characteristics of ionic liquids, such as dipolarity or polarizability, are correlated with their dissolution ability (Lungwitz et. al, New J. Chem, 2010, 34, 1135-1140). In addition to chemical diversity and novelty of potential ionic liquids, their safety and toxicity profile are of great importance. In general, toxicity of ionic liquids is influenced by the length of the cation alkyl side chain and the nature of the anion combination. Aquatic toxicity data for ionic liquids belonging to the 1-alkyl-3-methylimidazolium (MIM) class towards the marine bacterium Vibrio fischeri is well documented in the literature (Ventura et. al., Exotoxicology and Environmental Safety 76, 2012, pp 162-168).

A quantitative structure-function relationship (QSFR) model that correlates the activity of ionic liquids with their chemical structures has been developed. Molecular similarity searches using ionic liquids with plaque dissolution properties as input queries have been performed and public domain databases of compounds with a known safety profiles interrogated. A quantitative structure-function relationship (QSFR) model has now been developed to seek correlations between compound properties and their ability to dissolve biofilm. The compound properties considered are a combination of physicochemical properties, including molecular interaction, stereo-chemical and reactivity descriptors. Based on the results of the QSFR model, the property range for ionic liquids with biofilm removal data, encompassing atomic polarizability, kierflex and SMR descriptors, has been used as a benchmark for structure-function characterization comparisons.

Four ionic liquids have been used as “first tier” ionic liquids for input to molecular similarity search methodology RAMS (Rapid Assessment of Molecular Similarity). These four compounds are MMMPz MeSO4 (trimethylpyrazolium methylsulphate), EMIM EtSO4 (1-ethyl-3-methylimidazolium ethylsulphate), choline salicylate and TMIM MeSO4 (trimethylimidazolium methylsulphate). By using this RAMS approach, it has been possible to identify compounds that are similar to the query molecules in terms of shape, electrostatic potential and lipophilicity but with a different molecular skeleton.

Quantitative Structure Function Relationships (QSFR) models are used to build numerical models for molecules with experimental data for prediction and interpretation purposes. In order to build numerical models, it is necessary to represent molecular properties in the form of descriptors. A descriptor can be any quantity that depends on or describes the molecule, such as molecular weight, dipole moment or number of carbon or nitrogen or oxygen atoms. The QSFR model is an equation constructed from a set of molecules (generally referred to as the training set) where a subset (or vector) of the molecular descriptors is used to correlate with experimental results. In general, a linear regression or binary classification method is used to represent the models. The quality of the model can be reported in statistical terms, for example a correlation coefficient or percent accurate term. It is an underlying assumption of the model-building process that the training set is representative of a large sample of molecules, which can be modelled with independent and identically distributed uncorrelated variables. Model quality can be assessed in terms of its predictive ability, usually indicated by its ability to reproduce known experimental results of a test set of compounds (not used in model construction). When data is limited, cross-validation, a procedure by which portions of the training set are left out of the model building and subsequently used to simulate new molecules, is an alternative approach.

The process of QSFR model building involves assembling a dataset with experimental results and molecular structures, descriptor calculation and contingency analysis followed by model generation and validations. Ionic liquids with biofilm removal data served as a data set for model generation. The details of the dataset are shown in Table 1 (ID and biofilm removal data for ionic liquids selected for model generation (training set). For MMPZ MeSO₄ and EMIM Tosylate, where two values are available for biofilm removal, the value obtained at the higher pH value was considered for modelling work.

TABLE 1

| ID | % removal of biofilm |
|---|---|
| EMIM Cl | 44.3 |
| BMIM Cl | 43.8 |
| AMIM Cl | 44.8 |
| DMIM CL | 57.3 |
| EMIM Br | 48.6 |
| BMIM MeSO4 | 53.9 |
| TMIM MeSO4 | 51.8 |
| BMIMOAc | 55.9 |
| EMIM OAc | 52.1 |
| MTEOA MeSO4 | 52.8 |
| BMIM Br | 48.0 |
| MMMPz MeSO4 | 57.8 |
| EMIM Ethyl SO4 | 48.1 |
| BMIM Octyl SO4 | 58.6 |
| EMIM diethyl PO4 | 52.7 |
| EMIM Tosylate | 59.5 |

1-ethyl-3-methylimidazolium (EMIM) chloride (Cl), EMIM bromide (Br), EMIM ethyl sulfate, EMIM diethyl phosphate, EMIM acetate (OAc), EMIM tosylate, 1-butyl-3-methylimidazolium (BMIM) chloride (Cl), BMIM bromide (Br), BMIM methyl sulfate, BMIM octyl sulfate, BMIM acetate (OAc), 1-allyl-3-methylimidazolium (AMIM) chloride, 1-decyl-3-methylimidazolium (DMIM) chloride, 1,2,3-trimethylimidazolium methyl sulfate, 1,2,4-trimethylpyrazolium (MMMPZ) methyl sulfate, and tris (2-hydroxyethyl)methylammonium methylsulfate (MTEOA) methyl sulfate.

A number of descriptors representing molecular interaction, stereochemical and reactivity properties were calculated for the dataset of molecules. The descriptor calculation considered both cationic and anionic components of the ionic liquid.

The descriptors apol, weight, SMR, kierflex and PMI (atomic polarizability, molecular weight, molar refractivity, Kier flexibility index and principle moment of inertia) were found to correlate with biofilm removal data and were selected for model generation and validation. (The Kier flexibility index for a compound can be calculated from the Kier shape indices (¹κ_{α}—which encodes information about the count of atoms and relative cyclicity of molecules and ²κ_{α}—which encodes information about branching or relative spatial density of molecules and the number of non-hydrogen atoms in the molecule (N_{SA}) using the formula:

Φ=(¹κ_{α}²↓_{α})N_{SA}

### see L. B. Kier, Computational Chemical Graph Theory, D. H. Rouvray (Ed.), Nova Science Publishers, New York (1990)); Todeschini et al., Handbook of Molecular Descriptors, John Wiley & Sons, pgs 177-178, 248-250 (2008).

The QSFR model was generated by the partial least squares regression vector method to seek correlations between biofilm removal data for 16 ionic liquids and the four descriptors apol, weight, SMR, kierflex and PMI selected from the contingency analysis. For model validation purposes root mean square errors (RMSE) values and r² (the correlation coefficient, which lies between 0 and 1, with 1 corresponding to an ideal fit) were considered. The selected QSFR model is:

Activity=31.95+0.32(apol)+0.06(weight)−0.80(KierFlex)+0.22(SMR)

Correlation coefficient,r²=0.65

The biofilm removal capability is positively correlated to atomic polarizability, molecular refractivity and molecular weight. Conformational flexibility, as represented by the kierflex descriptor, is negatively correlated to biofilm removal capability. Thus we would predict that more conformationally flexible compounds would be less active.

In addition to the correlation coefficient r², measure, the QSFR model was evaluated for its ability to predict the activities of the molecules in the training set. The actual and predicted activity correlated within 5% of each other. Because of the limited size of the dataset, the “leave one out” (LOO) cross validation procedure was used for model evaluation. The correlation coefficient for cross validated predicted activity r² is 0.35.

Three descriptors (apol, SMR, kierflex) were identified from the QSFR model. Their property range for the 16 ionic liquids with biofilm removal data in the training set and the four ‘first tier’ ionic liquids as being 1-ethyl-3-imidazolidium (EMIM), 1,2,4-trimethylpyrazolium (MMMPz), tris(2-hydroxyethyl)methylammonium (Tris-HMAM) and choline. As can be seen, the bulk of the ionic liquids have properties within relatively narrow ranges (30-40 for apol, 5-15 for kierflex and 5-7 for SMR). Comparison of the descriptors apol, SMR, kierflex identified from the QSFR model property profile in known active ionic liquids with the property profile of potential ionic liquids has allowed alternative active compounds to be identified.

RAMS-ES (Rapid Assessment of Molecular Similarity-ElectroShape method (InhibOx) was used for molecular similarity search and selections. ElectroShape represents the shape and electrostatics of a molecule as a descriptor that is a compact string of 15 numbers generated from an analysis of interatomic distances and atomic partial charges. Molecules that are similar to a query molecule can be identified by comparison of their ElectroShape descriptors. ElectroShape was used to perform searches across Scopius-CSpace, a database of commercially available small molecule compounds (InhibOx).

First, conformational models for reference compounds were generated. Electroshape descriptors for selected conformations were then generated and CSPACE searched for the most similar structures. A visual selection of interesting matches was then made.

The input queries were based on low energy conformation for each of the four “first tier” ionic liquids and these were used to provide the input geometries. These ionic liquids were 1-ethyl-3-imidazolidium (EMIM), 1,2,4-trimethylpyrazolium (MMMPz), tris(2-hydroxyethyl)methylammonium (Tris-HMAM) and choline.

The cationic component were then enumerated with different anion combinations for subsequent structure function characterization. Chloride, bromide, acetate and methylsulphate anions were selected. In addition, functional groups that increase polarity of the ionic liquids into the sidechain of the cationic component were also proposed. A sidechain length limited to 4 carbon atoms was chosen to avoid possible toxicity due to an increase in hydrophibicity of the ionic liquid.

This has allowed new compounds for use in oral care compositions to be identified. The cations identified may be combined with different anions including chloride, bromide, acetate and sulphate.

The hits identified from molecular similarity searches were evaluated. Briefly, the highly similar cations (using the RAMS-ES similarity measure) were enumerated with four different anion combinations—chloride, bromide, acetate and sulphate. In the cases of imidazolium, pyrazolium and TRIS ammonium cations, compounds with sidechain modifications were also considered for enumeration with the four anions. The property profile of the potential ionic liquid combinations identified from each of the molecular similarity searches were then compared to the property profile described above.

The properties of the four “first tier” compounds is described below in Table 2.

TABLE 2

| Compound | apol | KierFlex | SMR |
|---|---|---|---|
| EMIM ethyl sulfate | 33 | 6 | 5 |
| MMMPz methyl sulfate | 30 | 5 | 5 |
| Choline salicylate | 37 | 5 | 6 |
| Tris-HMAM methyl sulfate | 38 | 14 | 6 |

### 1-Ethyl-3-methylimidazolium (EMIM) analogues

Structure function property profiles for potential ionic liquids identified from molecular similarity searches and side chain modifications of the 1-ethyl-3-methylimidazolium query are shown in Table 3. The property profile for the potential ionic liquids is comparable to the property profile of the four ‘first tier’ compounds. (MIM—3-methylmidazolium)

TABLE 3

| Compound | apol | KierFlex | SMR |
|---|---|---|---|
| EMIM ethyl sulfate | 33 | 6 | 5 |
| EMIM acetate | 26 | 4 | 5 |
| 1-OHEt-MIM BF₄ | 26 | 5 | 4 |
| 1-OHEt-MIM chloride | 23 | 4 | 3 |
| 1-OHEt-MIM bromide | 24 | 5 | 3 |
| 1-OHEt-MIM acetate | 28 | 4 | 4 |
| 1-OHEt-MIM sulfate | 31 | 7 | 5 |
| 1-OHPr-MIM chloride | 26 | 5 | 4 |
| 1-OHPr-MIM bromide | 27 | 6 | 4 |
| 1-OHPr-MIM acetate | 31 | 5 | 5 |
| 1-OHPr-MIM sulfate | 34 | 7 | 5 |
| 1-Me-3(4-OH—Bu)-imidazolium chloride | 29 | 6 | 4 |
| 1-Me-3(4-OH—Bu)-imidazolium bromide | 30 | 7 | 4 |
| 1-Me-3(4-OH—Bu)-imidazolium acetate | 34 | 6 | 5 |
| 1-Me-3(4-OH—Bu)-imidazolium sulfate | 36 | 8 | 6 |
| 1-Me-3(2-PrOEt)-imidazolium chloride | 32 | 5 | 5 |
| 1-Me-3(2-PrOEt)-imidazolium bromide | 33 | 6 | 5 |
| 1-Me-3(2-PrOEt)-imidazolium acetate | 40 | 6 | 6 |
| 1-Me-3(2-PrOEt)-imidazolium sulfate | 43 | 8 | 7 |

### 1,2,4-Trimethylpyrazolium (MMMPz) analogues

Structure function property profile for potential ionic liquids identified from molecular similarity searches and side chain modifications of the 1,2,4-Trimethylpyrazolium query are shown in Table 4. Property profiles for many of the potential ionic liquids are comparable to the property profile of the four ‘First Tier’ compounds. (Pz—pyrazolium)

TABLE 4

| Compound | apol | KierFlex | SMR |
|---|---|---|---|
| MMMPz methyl sulfate | 30 | 5 | 5 |
| 1,2-dimethyl-4-propyl-Pz sulfate | 30 | 5 | 5 |
| MMMPz chloride | 22 | 3 | 3 |
| MMMPz bromide | 23 | 3 | 3 |
| MMMPz acetate | 27 | 3 | 4 |
| 1,2-dimethyl-4-ethyl-Pz methyl sulfate | 33 | 6 | 5 |
| 1,2-dimethyl-4-ethyl-Pz chloride | 25 | 3 | 3 |
| 1,2-dimethyl-4-ethyl-Pz | 26 | 4 | 3 |
| 1,2-dimethyl-4-ethyl-Pz acetate | 30 | 4 | 5 |
| 1,2-dimethyl-4-ethyl-Pz sulfate | 36 | 7 | 6 |
| 1,2-dimethyl-4-propyl-Pz chloride | 28 | 4 | 4 |
| 1,2-dimethyl-4-propyl-Pz bromide | 29 | 5 | 4 |
| 1,2-dimethyl-4-propyl-Pz acetate | 34 | 4 | 5 |
| 1,2-dimethyl-4-butyl-Pz sulfate | 39 | 7 | 6 |
| 1,2-dimethyl-4-butyl-Pz chloride | 32 | 5 | 5 |
| 1,2-dimethyl-4-butyl-Pz bromide | 32 | 6 | 5 |
| 1,2-dimethyl-4-butyl-Pz acetate | 36 | 5 | 6 |

### Choline Analogues

Structure function property profile for potential ionic liquids identified from molecular similarity searches and sidechain modifications of the choline query with similar property profiles similar to the first compounds are shown in Table 5 (see next page).

TABLE 5

| Compound | apol | KierFlex | SMR |
|---|---|---|---|
| Choline salicylate | 37 | 5 | 6 |
| Choline bitartrate | 35 | 8 | 5 |
| Choline sulfate | 30 | 10 | 5 |
| Choline bromide | 23 | 10 | 3 |
| (2-chloro-3-hydroxypropyl)trimethylammonium | 24 | 10 | 3 |
| chloride | | | |
| (2-chloro-3-hydroxypropyl)trimethylammonium | 25 | 12 | 3 |
| bromide | | | |
| (2-chloro-3-hydroxypropyl)trimethylammonium | 29 | 9 | 5 |
| acetate | | | |
| (2-chloro-3-hydroxypropyl)trimethylammonium | 31 | 12 | 5 |
| sulfate | | | |
| 4-(2-hydroxyethyl)-1,1-dimethylpiperazin-1- | 32 | 6 | 5 |
| ium chloride | | | |
| 4-(2-hydroxyethyl)-1,1-dimethylpiperazin-1- | 33 | 7 | 5 |
| ium bromide | | | |
| 4-(2-hydroxyethyl)-1,1-dimethylpiperazin-1- | 37 | 6 | 6 |
| ium acetate | | | |
| 4-(2-hydroxyethyl)-1,1-dimethylpiperazin-1- | 40 | 9 | 6 |
| ium sulfate | | | |
| (2,3-dihydroxypropyl) trimethylammonium | 26 | 8 | 6 |
| chloride | | | |
| (2,3-dihydroxypropyl) trimethylammonium | 27 | 10 | 4 |
| bromide | | | |
| (2,3-dihydroxypropyl) trimethylammonium | 31 | 7 | 4 |
| acetate | | | |
| (2,3-dihydroxypropyl) trimethylammonium | 34 | 11 | 5 |
| sulfate | | | |
| hydrogen (3-cyano-2-hydroxypropyl) | 28 | 7 | 4 |
| trimethylazanium chloride | | | |
| hydrogen (3-cyano-2-hydroxypropyl) | 28 | 8 | 4 |
| trimethylazanium bromide | | | |
| hydrogen (3-cyano-2-hydroxypropyl) | 32 | 7 | 5 |
| trimethylazanium acetate | | | |
| hydrogen (3-cyano-2-hydroxypropyl) | 35 | 9 | 6 |
| trimethylazanium sulfate | | | |

### Tris(2-hydroxyethyl)methylammonium (Tris(2-hydroxyethyl MEA) analogues

Potential ionic liquids identified from molecular similarity searches and sidechain modifications of Tris(2-hydroxyethyl)methylammonium query, structure function property profiles are shown in Table 6 (see next page). (MEA=methylammonium)

TABLE 6

| Compound | apol | KierFlex | SMR |
|---|---|---|---|
| Tris-(2-hydroxyethyl) MEA methyl | 38 | 14 | 6 |
| sulfate | | | |
| Tris-(2-hydroxyethyl) MEA methyl | 30 | 12 | 4 |
| chloride | | | |
| Tris-(2-hydroxyethyl) MEA methyl | 31 | 14 | 4 |
| bromide | | | |
| Tris-(2-hydroxyethyl) MEA methyl | 35 | 11 | 5 |
| acetate | | | |
| Bis-(2-hydroxypropyl)-2- | 43 | 16 | 6 |
| hydroxyethyl MEA methyl sulfate | | | |
| Bis-(2-hydroxypropyl)-2- | 36 | 14 | 5 |
| hydroxyethyl MEA chloride | | | |
| Bis-(2-hydroxypropyl)-2- | 37 | 16 | 5 |
| hydroxyethyl MEA bromide | | | |
| Bis-(2-hydroxypropyl)-2- | 41 | 13 | 6 |
| hydroxyethyl MEA acetate | | | |
| Bis-(2-hydroxybutyl)-2-hydroxyethyl | 50 | 18 | 8 |
| MEA methyl sulfate | | | |
| Bis-(2-hydroxybutyl)-2-hydroxyethyl | 41 | 16 | 6 |
| MEA chloride | | | |
| Bis-(2-hydroxybutyl)-2-hydroxyethyl | 42 | 18 | 6 |
| MEA bromide | | | |
| Bis-(2-hydroxybutyl)-2-hydroxyethyl | 47 | 15 | 7 |
| MEA acetate | | | |
| Bis-(2-hydroxypentyl)-2- | 56 | 20 | 9 |
| hydroxyethyl MEA methyl sulfate | | | |
| Bis-(2-hydroxypentyl)-2- | 49 | 18 | 7 |
| hydroxyethyl MEA chloride | | | |
| Bis-(2-hydroxypentyl)-2- | 49 | 20 | 7 |
| hydroxyethyl MEA bromide | | | |
| Bis-(2-hydroxybutyl)-2-hydroxyethyl | 54 | 17 | 8 |
| MEA acetate | | | |

### Divalent Cation Analogues

RAMS-ES searches with the Tris(2-hydroxyethyl)methylammonium query identified a number of compounds with two cationic centres belonging to morpholonium, piperzinium, pyrrolium and ammonium chemical classes. These divalent cations were enumerated with divalent anion combinations (chloride, bromide, acetate and sulphate) and their property profiles compared as above. The structure function property profiles are shown in Table 7.

TABLE 7

| Compound | apol | KierFlex | SMR |
|---|---|---|---|
| 2-(2-methylmorpholin-4-yl)ethan-1- | 32 | 15 | 4 |
| amine di-chloride | | | |
| 2-(2-methylmorpholin-4-yl)ethan-1- | 33 | 21 | 4 |
| amine di-bromide | | | |
| 2-(2-methylmorpholin-4-yl)ethan-1- | 42 | 12 | 6 |
| amine di-acetate | | | |
| 2-(2-methylmorpholin-4-yl)ethan-1- | 47 | 18 | 7 |
| amine di-sulfate | | | |
| 1-(2-methoxyethyl)pyrrolidin-3- | 32 | 15 | 4 |
| amine di-chloride | | | |
| 1-(2-methoxyethyl)pyrrolidin-3- | 33 | 21 | 4 |
| amine di-bromide | | | |
| 1-(2-methoxyethyl)pyrrolidin-3- | 42 | 12 | 6 |
| amine di-acetate | | | |
| 1-(2-methoxyethyl)pyrrolidin-3- | 47 | 18 | 7 |
| amine di-sulfate | | | |
| 4-amino-3- | 33 | 22 | 4 |
| [(dimethylamino)methyl]butan-2-ol | | | |
| di-chloride | | | |
| 4-amino-3- | 35 | 31 | 4 |
| [(dimethylamino)methyl]butan-2-ol | | | |
| di-bromide | | | |
| 4-amino-3- | 43 | 16 | 6 |
| [(dimethylamino)methyl]butan-2-ol | | | |
| di-acetate | | | |
| 4-amino-3- | 48 | 22 | 7 |
| [(dimethylamino)methyl]butan-2-ol | | | |
| di-sulfate | | | |
| 3-(morpholin-4-yl)propan-1-amine | 32 | 18 | 4 |
| di-chloride | | | |
| 3-(morpholin-4-yl)propan-1-amine | 33 | 25 | 4 |
| di-bromide | | | |
| 3-(morpholin-4-yl)propan-1-amine | 42 | 14 | 6 |
| di-acetate | | | |
| 3-(morpholin-4-yl)propan-1-amine | 47 | 20 | 7 |
| di-sulfate | | | |
| 3-(2-methylpiperaziny-1-yl)propan-1- | 35 | 16 | 4 |
| ol di-chloride | | | |
| 3-(2-methylpiperaziny-1-yl)propan-1- | 37 | 22 | 4 |
| ol di-bromide | | | |
| 3-(2-methylpiperaziny-1-yl)propan-1- | 45 | 13 | 6 |
| ol di-acetate | | | |
| 3-(2-methylpiperaziny-1-yl)propan-1- | 50 | 19 | 8 |
| ol di-sulfate | | | |

### Pyridinium Analogues

Using the approach of the previous section, the characterization of pyridinium-containing ionic liquids with the cationic components has been performed with four different anions chloride, bromide, acetate and sulphate. The results are shown in Table 8.

TABLE 8

| Compound | apol | KierFlex | SMR |
|---|---|---|---|
| 1-butyl pyridinium chloride | 28 | 5 | 4 |
| 1-butyl pyridinium bromide | 29 | 7 | 4 |
| 1-butyl pyridinium acetate | 33 | 6 | 5 |
| 1-butyl pyridinium methyl sulfate | 36 | 8 | 6 |
| 1-butyl-4-methyl pyridinium chloride | 32 | 5 | 5 |
| 1-butyl-4-methyl pyridinium bromide | 32 | 6 | 5 |
| 1-butyl-4-methyl pyridinium acetate | 36 | 6 | 6 |
| 1-butyl-4-methyl pyridinium methyl | 39 | 8 | 6 |
| sulfate | | | |
| 1-butyl-3-methyl pyridinium chloride | 32 | 6 | 5 |
| 1-butyl-3-methyl pyridinium bromide | 32 | 6 | 5 |
| 1-butyl-3-methyl pyridinium acetate | 36 | 6 | 6 |
| 1-butyl-3-methyl pyridinium methyl | 39 | 8 | 6 |
| sulfate | | | |

### Pyrrolidium Analogues

Two sets of side chain modifications were considered for pyrrolidium based cationic components:

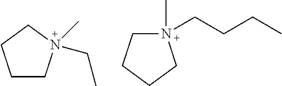

These analogues were enumerated with seven different anion combinations—chloride, bromide, iodide, PF6, BF4, acetate and sulphate. The results are shown in Table 9.

TABLE 9

| Compound | apol | KierFlex | SMR |
|---|---|---|---|
| N-ethyl-N-methyl pyrrolidium chloride | 26 | 5 | 4 |
| N-ethyl-N-methyl pyrrolidium bromide | 27 | 6 | 4 |
| N-ethyl-N-methyl pyrrolidium | 31 | 4 | 5 |
| hexafluorophosphate | | | |
| N-ethyl-N-methyl pyrrolidium BF₄ | 29 | 5 | 5 |
| N-ethyl-N-methyl pyrrolidium acetate | 31 | 5 | 5 |
| N-ethyl-N-methyl pyrrolidium methyl | 34 | 7 | 5 |
| sulfate | | | |
| N-butyl-N-methyl pyrrolidium chloride | 32 | 6 | 4 |
| N-butyl-N-methyl pyrrolidium bromide | 33 | 8 | 4 |
| N-butyl-N-methyl pyrrolidium acetate | 37 | 6 | 5 |
| N-butyl-N-methyl pyrrolidium methyl | 40 | 9 | 6 |
| sulfate | | | |
| N-butyl-N-methyl pyrrolidium iodide | 36 | 9 | 4 |
| N-butyl-N-methyl pyrrolidium BF₄ | 36 | 7 | 6 |
| N-butyl-N-methyl pyrrolidium | 37 | 5 | 6 |
| hexafluorophosphate | | | |

Other Salt Compounds with Requisite Properties

Other salt compounds may be suitable substitutes for the above ionic liquids based on their atomic polarizability (apol), KierFlex and molar refractivity values. Suitable compounds include, but are not limited to:

| Compound | apol | KierFlex | SMR |
|---|---|---|---|
| Ferric ammonium citrate | 32 | 11 | 3 |
| Ferrous ascorbate | 48 | 11 | 7 |
| Calcium L-ascorbate | 63 | 9 | 7 |
| Biotin | 35 | 4 | 6 |
| Butylated hydroxyanisole | 32 | 2 | 5 |
| Triethyl citrate | 40 | 7 | 6 |

One embodiment of the invention is to select compounds with known safety and toxicity profiles. By integrating available safety information, a prioritized selection of 15 compounds has been selected:
(2-chloro-3-hydroxypropyl)trimethylammonium chloride;
(2,3-dihydroxypropyl)trimethylammonium chloride;
1-butyl pyridinium bromide;
1-butyl-4-methyl pyridinium chloride;
1-butyl-4-methyl pyridinium bromide;
1-butyl-4-methyl pyridinium iodide;
1-butyl-4-methyl pyridinium BF₄;
1-butyl-4-methyl pyridinium hexafluorophosphate;
N-butyl-N-ethyl pyrrolidium hexafluorophosphate;
N-butyl-N-ethyl pyrrolidium BF₄;
N-butyl-N-methyl pyrrolidium chloride;
N-butyl-N-methyl pyrrolidium bromide;
N-butyl-N-methyl pyrrolidium iodide;
N-butyl-N-methyl pyrrolidium BF₄;
N-butyl-N-methyl pyrrolidium hexafluorophosphate.

This prioritized selection includes two ammonium based ionic liquids with a polar side chain with the aim of reducing hydrophobicity and potential toxicity. The remaining compounds in the list belong to pyridinium or pyrrolidinium cationic compound classes.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the scope of the invention should be construed broadly as set forth in the appended claims.

In some embodiments the compounds of the present invention are present in the oral care composition in an amount of about 0.1 wt % to about 30 wt % based on the total weight of the composition. In some embodiments, the compound is present in the composition in an amount of about 0.5 wt % to about 20 wt % based on the total weight of the composition. In some embodiments, the compound is present in the composition in an amount of about 5 wt % to about 15 wt % based on the total weight of the composition. In some embodiments, the compound is present in the composition in an amount of about 8 wt % to about 10 wt % based on the total weight of the composition.

In some embodiments, the compound is an ionic liquid.

In some embodiments, in the compound is present in the composition at a concentration of about 1 mM to about 500 mM. In some embodiments, the compound is present in the composition at a concentration of about 5 mM to about 300 mM. Further optionally, the compound is present in the composition at a concentration of about 15 mM to about 250 mM or about 1 mM to about 50 mM.

In some embodiments, the oral care composition of the present invention will comprise ingredients in addition to the compound of the present invention.

In some embodiments, the composition comprises an orally acceptable carrier for a mouth rinse, toothpaste, oral beads or strips, irrigation fluid, plaque removal liquid, tongue spray, dental floss, candy, lozenge, chewing gum, and lollipop.

In some embodiments, the composition further comprises one or more agents selected from diluents, bicarbonate salts, pH modifying agents, surfactants, foam modulators, thickening agents, viscosity modifiers, humectants, sweeteners, flavorants, pigments, anticaries agents, anticalculus or tartar control agents, abrasives and mixtures thereof

### Anions

In some embodiments, the anion is a halide. As used herein, the term “halide” refers to Fl, Cl, Br, I. In some embodiments, the anion is a halide selected from Br and Cl. In some embodiments, the anion is an alkyl sulfate selected from methyl sulfate, ethyl sulfate, propyl sulfate, butyl sulfate, pentyl sulfate, hexyl sulfate, heptyl sulfate, and octyl sulfate. In some embodiments, the alkyl sulfate and alkyl phosphate comprise from 1 to 22 carbon atoms. Preferably, the alkyl sulfate and alkyl phosphate comprise from 1 to 4 carbon atoms 6 to 10, or from 12 to 22 carbon atoms.

In some embodiments, the anion is selected from the group consisting of acetate, bromide, chloride, methyl sulfate, ethyl sulfate, octyl sulfate, diethyl phosphate, and tosylate. In some embodiments, the anion is selected from the group consisting of acetate, octylsulfate or tosylate. In one embodiment, the anion is bromide. In a further embodiment, the anion is tosylate. In a yet further embodiment, the anion is acetate.

In some embodiments, the compound is present in the oral care composition in an amount of about 0.1 wt % to about 30 wt % based on the total weight of the composition. In some embodiments, the compound is present in the oral care composition in an amount of about 0.5 wt % to about 20 wt %, or from about 1 wt % to about 15 wt %, based on the total weight of the composition. In some embodiments, the compound is present in the oral care composition in an amount of about 5 wt % to about 15 wt %, or from about 7 wt % to about 12 wt %, based on the total weight of the composition. In some embodiments the compound is present in the oral care composition in an amount of about 8 wt % to about 10 wt % based on the total weight of the composition.

In some embodiments, the compound is present in the oral care composition in a concentration of about 1 mM to about 500 mM, or from about 4 mM to about 400 mM. In some embodiments the compound is present in the oral care composition in a concentration of about 5 mM to about 300 mM or from about 100 mM to about 270 mM. In some embodiments, the compound is present in the oral care composition in a concentration of about 15 mM to about 250 mM, or from about 18 mM to about 220 mM or about 1 mM to about 50 mM.

### Abrasives

Whilst the compositions of the present invention may optionally further comprise an abrasive which may be useful, for example, as a polishing agent, it has been found that oral care compositions comprising compounds as defined herein are effective in removing biofilm or plaque, and whitening teeth, without the need for substantial amounts of abrasives. This is advantageous because abrasives can damage enamel and expose dentine tissues with repeated use, particularly, in subjects with soft enamel caused by disease or excessive exposure to food acids.

In one embodiment, the oral care composition comprises an abrasive in an amount of less than 0.1 wt % by total weight of the composition. In another embodiment, the oral care composition comprises an abrasive in an amount of less than 0.01 wt % by total weight of the composition. In yet another embodiment, the composition is substantially free, or free, of any abrasives.

Suitable optional abrasives include silica, for example in the form of precipitated silica or as admixed with alumina, insoluble phosphates, calcium carbonate, and mixtures thereof. Among insoluble phosphates useful as abrasives are orthophosphates, polymetaphosphates and pyrophosphates. Illustrative examples are dicalcium orthophosphate dihydrate, calcium pyrophosphate, calcium pyrophosphate, tricalcium phosphate, calcium polymetaphosphate and insoluble sodium polymetaphosphate.

### Carrier

Among useful carriers for optional inclusion in a composition of the invention are diluents, bicarbonate salts, pH modifying agents, surfactants, foam modulators, thickening agents, viscosity modifiers, humectants, sweeteners, flavorants, pigments, anticaries agents, and anticalculus or tartar control agents, abrasives and mixtures thereof. Carriers should be selected for compatibility with each other and with other ingredients of the composition.

Water is a preferred diluent and in some compositions such as mouthwashes, water is commonly accompanied by an alcohol, e.g., ethanol. The weight ratio of water to alcohol in a mouthwash composition is generally 1:1 to 20:1, for example 3:1 to 20:1 or 4:1 to 10:1. In a whitening liquid, the weight ratio of water to alcohol can be within or below the above ranges, for example, 1:10 to 2:1.

### Optional Oral Care Ingredients

In a further embodiment, the composition of the invention comprises at least one bicarbonate salt, useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. One or more bicarbonate salts are optionally present in a total amount of about 0.1 wt % to about 50 wt %, for example about 1 wt % to 20 wt %, by total weight of the composition.

In a still further embodiment, the composition of the invention comprises at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and the like. One or more pH modifying agents are optionally present in a total amount effective to maintain the composition in an orally acceptable pH range.

In a still further embodiment, the composition of the invention comprises at least one surfactant. Any orally acceptable surfactant, most of which are anionic, nonionic or amphoteric, can be used. Suitable anionic surfactants include without limitation, water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation, derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of about 0.01 wt % to about 10 wt %, for example, from about 0.05 wt % to about 5 wt %, or from about 0.1 wt % to about 2 wt % by total weight of the composition.

In a still further embodiment, the composition of the invention comprises at least one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000,000 to 2,500,000. One or more PEGs are optionally present in a total amount of about 0.1 wt % to about 10 wt %, for example from about 0.2 wt % to about 5 wt %, or from about 0.25 wt % to about 2 wt %, by total weight of the composition.

In a still further embodiment, the composition of the invention comprises at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly t-carrageenan (iota-carrageenan), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol® series. Particularly preferred Carbopols include Carbopol 934, 940, 941, 956, 974P, and mixtures thereof. One or more thickening agents are optionally present in a total amount of from about 0.01 wt % to 15 wt %, for example from about 0.1 wt % to about 10 wt %, or from about 0.2 wt % to about 5 wt %, by total weight of the composition.

In a still further embodiment, the composition of the invention comprises at least one viscosity modifier, useful for example to inhibit settling or separation of ingredients or to promote re-dispersibility upon agitation of a liquid composition. Any orally acceptable viscosity modifier can be used, including without limitation, mineral oil, petrolatum, clays and organomodified clays, silica and the like. One or more viscosity modifiers are optionally present in a total amount of from about 0.01 wt % to about 10 wt %, for example, from about 0.1 wt % to about 5 wt %, by total weight of the composition.

In a still further embodiment, the composition of the invention comprises at least one humectant. Any orally acceptable humectant can be used, including without limitation, polyhydric alcohols such as glycerin, sorbitol, xylitol or low molecular weight PEGs. Most humectants also function as sweeteners. One or more humectants are optionally present in a total amount of from about 1 wt % to about 70 wt %, for example, from about 1 wt % to about 50 wt %, from about 2 wt % to about 25 wt %, or from about 5 wt % to about 15 wt %, by total weight of the composition.

In a still further embodiment, a composition of the invention comprises at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount depending strongly on the particular sweetener(s) selected, but typically 0.005 wt % to 5 wt %, by total weight of the composition.

In a still further embodiment, a composition of the invention comprises at least one flavorant, useful for example to enhance taste of the composition. Any orally acceptable natural or synthetic flavorant can be used, including without limitation vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt % to about 5 wt %, for example, from about 0.1 wt % to about 2.5 wt %, by total weight of the composition.

In a still further embodiment, a composition of the invention may comprise at least one colorant. Colorants herein include pigments, dyes, lakes and agents imparting a particular luster or reflectivity such as pearling agents. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like. One or more colorants are optionally present in a total amount of from about 0.001 wt % to about 20 wt %, for example, from about 0.01 wt % to about 10 wt %, or from about 0.1 wt % to about 5 wt %, by total weight of the composition.

In some embodiments, the composition comprises a fluoride ion source. Fluoride ion sources include, but are not limited to: stannous fluoride, sodium fluoride, potassium fluoride, potassium monofluorophosphate, sodium monofluorophosphate, ammonium monofluorophosphate, sodium fluorosilicate, ammonium fluorosilicate, amine fluoride such as olaflur (N′-octadecyltrimethylendiamine-N,N,N′-tris(2-ethanol)-dihydrofluoride), ammonium fluoride, and combinations thereof. In certain embodiments the fluoride ion source includes stannous fluoride, sodium fluoride, amine fluorides, sodium monofluorophosphate, as well as mixtures thereof. In certain embodiments, the oral care composition of the invention may also contain a source of fluoride ions or fluorine-providing ingredient in amounts sufficient to supply about 50 to about 5000 ppm fluoride ion, e.g., from about 100 to about 1000, from about 200 to about 500, or about 250 ppm fluoride ion. Fluoride ion sources may be added to the compositions of the invention at a level of about 0.001 wt % to about 10 wt %, e.g., from about 0.003 wt % to about 5 wt %, 0.01 wt % to about 1 wt, or about 0.05 wt %. However, it is to be understood that the weights of fluoride salts to provide the appropriate level of fluoride ion will obviously vary based on the weight of the counter ion in the salt, and one of skill in the art may readily determine such amounts. A preferred fluoride salt may be sodium fluoride.

The composition of the present invention optionally comprises a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

The composition of the present invention optionally incorporates one or more antisensitivity agents, e.g., potassium salts such as potassium nitrate, potassium bicarbonate, potassium chloride, potassium citrate, and potassium oxalate; capsaicin; eugenol; strontium salts; zinc salts; chloride salts and combinations thereof. Such agents may be added in effective amounts, e.g., from about 1 wt % to about 20 wt % by weight based on the total weight of the composition, depending on the agent chosen. The compositions of the present invention may also be used to treat hypersensitivity by blocking dentin tubules when applied to a tooth.

In some embodiments, the composition of the invention further comprises an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

In another embodiment, the composition comprises an orally acceptable zinc ion source useful, for example, as an antimicrobial, anticalculus or breath-freshening agent. One or more such sources can be present. Suitable zinc ion sources include without limitation zinc acetate, zinc citrate, zinc gluconate, zinc glycinate, zinc oxide, zinc sulfate, sodium zinc citrate and the like. One or more zinc ion sources are optionally and illustratively present in a total amount of from about 0.05 wt % to about 3 wt %, for example from about 0.1 wt % to about 1 wt %, by total weight of the composition.

The composition of the present invention may additionally optionally comprise a tartar control (anticalculus) agent as provided below. Tartar control agents among those useful herein include salts of the specified agents, including alkali metal and ammonium salts. The agents include: phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof. Other useful tartar control agents include polycarboxylate polymers and polyvinyl methyl ether/maleic anhydride (PVM/MA) copolymers, such as GANTREZ®.

In some embodiments, the composition of the present invention further comprises a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophan, L-lysine, methionine, threonine, levocarnitine and L-carnitine), lipotropics (such as choline, inositol, betaine, and linoleic acid), and mixtures thereof

In some embodiments, the oral care composition of the invention does not contain any other antibacterial or whitening agent.

### Delivery

The oral care composition of the present invention preferably comprises an orally acceptable carrier for use in a product such as a mouth rinse (including dual phase mouthwash), toothpaste, actives in beads/strips, irrigation fluids, plaque removal fluids, Wisp® formulas, formulations to be delivered through devices such as pens, back of a toothbrush and front of a toothbrush, formulations to be delivered through porous wicking materials, interdental brushes, fluid encased dental strips, floss impregnated or coated with the formulations or dried formulations, portables, oral trays, hard or soft candy, lozenge with a soft plaque dissolving liquid inside, lollipops with the plaque dissolving formulation imbedded into the lickable “candy” that can also help control tongue bacteria, peelable gels, patches, formulations for pop-rocks that upon popping, spread a fine mist of the formulation around oral cavity, tongue cleaners with plaque dissolving strips and dental strips. Accordingly, opportunities exist for professional use of the compositions of the present invention (e.g. during cleanings, irrigations, or aggressive periodontal procedures, such as root planning & scaling). The composition of the invention may be provided in any of the products defined herein. If used in animals or pets, veterinary pastes, chewables or treats may also be used as the orally acceptable carrier.

In one embodiment, the composition of the invention can be dried into powder and utilized in a portable sachet. For example, upon mixing such a powder with a suitable solvent such as water, a rinse may be created to remove plaque, proteins and other debris in the mouth.

In another embodiment, the composition of the invention can be dried with abrasives such as silica, calcium carbonate or soft capsules that upon addition of small amount of water, creates a paste to brush away the plaque.

Formulations that increase the substantivity of compounds onto a surface could be expected to increase the efficacy of biofilm, and hence plaque removal. For example, Tween 20 while also functioning as a surfactant, is also a wetting agent. Therefore, incorporation of such an agent could be expected to increase the wettability and spreading of a mouth rinse formulation according to the present invention, over the soft and hard tissue, increasing the formulation's propensity for plaque dissolution and removal.

### Methods of Use

The composition according to the present invention may be administered to or applied to a human or other animal subject. The composition may be suitable for administration or application to the oral cavity of a human or animal subject. Typically, the composition is for reducing or removing dental plaque. The reduction or removal of plaque may occur through an inhibition of biofilm (a plaque precursor) formation and/or degradation of microbial biofilm.

The present invention further provides a composition as defined above for preventing or treating a disease condition of the oral cavity. Typically, the disease condition is caused by plaque. The disease condition may be selected from tooth decay, periodontal disease, gingivitis and xerostomia (dry mouth).

Accordingly, the present invention provides a composition as defined above for use as a medicament.

The present invention also provides a method of removing or reducing plaque from the oral cavity of a subject comprising administering a therapeutically effective amount of a composition comprising a compound as defined herein, and the composition is applied to the oral cavity.

In a preferred embodiment, the method is for treating or preventing a condition caused by plaque. Preferably, condition caused by plaque is selected from tooth decay, periodontal disease, gingivitis and xerostomia (dry mouth).

The present invention further provides a method of whitening teeth in a subject comprising administering a therapeutically effective amount of a composition comprising at least one ionic liquid to the subject. Preferably, the composition is an oral care composition comprising a compound as defined herein, and the composition is applied to the oral cavity.

The present invention still further provides a method of reducing the amount of bacteria in the oral cavity of a subject comprising administering a therapeutically effective amount of a composition comprising a compound as defined herein to the oral cavity.

The present invention additionally provides a use of an oral care composition for removing or reducing plaque in the oral cavity of a subject. The oral care composition is as defined herein.

The present invention further provides a use of an oral care composition for whitening teeth in the oral cavity of a subject. The oral care composition is as defined herein.

The present invention still further provides a use of an oral care composition, for reducing the amount of bacteria the oral cavity of a subject, wherein the oral care composition is as defined herein.

Compositions comprising a compound as defined herein possess the ability to offer a deep but gentle cleaning, and promote removal of biofilm and plaque without the need for harsh abrasives or rigorous brushing. The compositions are further able to remove stains and whiten teeth, again without the need for harsh abrasives or rigorous brushing.

1. An oral care composition comprising a compound, wherein the compound comprises:
(a) a cation; and
(b) an anion
wherein the compound has two or more of
(i) an atomic polarizability of from 20 to 60,
(ii) a Kier flexibility index of from 2 to 20 and
(iii) a molar refractivity of from 3 to 10.

2. The oral care composition comprising a compound, wherein the compound comprises:
(a) a cation comprising a positively charged nitrogen atom and
(b) an anion
wherein the compound has two or more of
(i) an atomic polarizability of from 20 to 60,
(ii) a Kier flexibility index of from 2 to 20 and
(iii) a molar refractivity of from 3 to 10.

3. The oral care composition according to claim 1 wherein the compound has an atomic polarizability of from 28 to 40.

4. The oral care composition according to claim 1 wherein the compound has a Kier flexibility index of from 4 to 15.

5. The oral care composition according to claim 1 wherein the compound has a molar refractivity of from 4 to 7.

6. The oral care composition according to claim 1 wherein the compound has two or more of
(i) an atomic polarizability of from 2.8 to 40,
(ii) a Kier flexibility index of from 4 to 15 and
(iii) a molar refractivity of from 4 to 7.

7. The oral care composition according to claim 1 wherein the compound has
(i) an atomic polarizability of from 30 to 38 and
(ii) a Kier flexibility index of from 5 to 14 and
(iii) a molar refractivity of from 5 to 6.

8. The oral care composition according to claim 1 wherein the compound is selected from one or more of EMIM ethyl sulfate; EMIM acetate; 1-OHEt-MIM BF₄; 1-OHEt-MIM chloride; 1-OHEt-MIM bromide; 1-OHEt-MIM acetate; 1-OHEt-MIM sulfate; 1-OHPr-MIM chloride; 1-OHPr-MIM bromide; 1-OHPr-MIM acetate; 1-OHPr-MIM sulfite; 1-Me-3(4-OH-Bu)-imidazolium chloride: 1-Me-3(4-OH-Bu)-imidazolium bromide; 1-Me-3 (4-O-Bu)-imidazolium acetate; 1-Me-3(4-OH-Bu imidazolium sulfate; 1-Me-3(2-PrOEt)-imidazolium chloride; 1-Me-3(2-PrOEt)-imidazolium bromide; 1-Me-3(2-PrOEt)-imidazolium acetate; 1-Me-3(2-PrOEt)-imidazolium sulfate; MMMPz methyl sulfate; 1,2-dimethyl-4-propyl-Pz sulfate; MMMPz chloride; MMMPz bromide; MMMPz acetate; 1,2-dimethyl-4-ethyl-Pz methyl sulfate 1,2-dimethyl-4-ethyl-Pz chloride; 1,2-dimethyl-4-ethyl-Pz; 1,2-dimethyl-4-ethyl-Pz acetate; 1,2-dimethyl-4-ethyl-Pz sulfate; 1,2-dimethyl-4-propyl-Pz chloride; 1,2-dimethyl-4-propyl-Pz bromide; 1,2-dimethyl-4-propyl-Pz acetate; 1,2-dimethyl-4-butyl-Pz sulfate; 1,2-dimethyl-4-butyl-Pz chloride 1,2-dimethyl-4-butyl-Pz bromide; 1,2-dimethyl-4-butyl-Pz acetate; Choline salicylate; Choline bitartrate; Choline sulfate; Choline bromide; (2-chloro-3-hydroxypropyl)trimethylammonium chloride; (2-chloro-3-hydroxypropyl)trimethylammonium bromide; (2-chloro-3-hydroxypropyl)trimethylammonium acetate; (2-chloro-3-hydroxypropyl)trimethylammonium sulfate; 2-hydroxyethyl)-1,1-dimethylpiperazin-1-ium chloride; 4-(2-hydroxyethyl)-1,1-di methylpiperazin-1-ium bromide; 4-(2-hydroxyethyl)-1,1-dimethylpiperazin-1-ium acetate; 4-(2-hydroxyethyl)-1,1-dimethylpiperazin-1-ium sulfate; (2,3-dihydroxypropyl) trimethylammonium chloride; (2,3-dihydroxypropyl)trimethylammonium bromide; (2,3-dihydroxypropyl)trimethylammonium acetate; (2,3-dihydroxypropyl)trimethylammonium sulfate; hydrogen (3-cyano-2-hydroxypropyl)trimethylazanium chloride; hydrogen (3-cyano-2-hydroxypropyl)trimethylazanium bromide: hydrogen (3-cyano-2-hydroxypropyl)trimethylazanium acetate; hydrogen (3-cyano-2-hydroxypropyl)trimethylazanium sulfate; Tris-(2-hydroxyethyl) MEA methyl sulfate; Tris-(2-hydroxyethyl) MEA methyl chloride; Tris-(2-hydroxyethyl) MEA methyl bromide; Tris-(2-hydroxyethyl) MEA methyl acetate; Bis-(2-hydroxypropyl)-2-hydroxyethyl MEA methyl sulfate; Bis-(2-hydroxypropyl)-2-hydroxyethyl MEA chloride; Bis-(2-hydroxypropyl)-2-hydroxyethyl MEA bromide; Bis-(2-hydroxypropyl)-2-hydroxyethyl MEA acetate; Bis-(2-hydroxybutyl)-2-hydroxyethyl MEA methyl sulfate; Bis-(2-hydroxybutyl)-2-hydroxyethyl MEA chloride; Bis-(2-hydroxybutyl)-2-hydroxy cal MEA bromide; Bis-(2-hydroxybutyl)-2-hydroxyethyl MEA acetate; Bis-(2-hydroxypentyl)-2-hydroxyethyl MEA methyl sulfate; Bis-(2-hydroxypentyl)-2-hydroxyethyl MEA chloride; Bis-(2-hydroxypentyl)-2-hydroxyethyl MEA bromide; Bis-(2-hydroxybutyl)-2-hydroxyethyl MEA acetate; 2-(2-methylmorpholin-4-yl)ethan-1-amine di-chloride; 2-(2-methylmorpholin-4-yl)ethan-1-amine di-bromide; 2-(2-methylmorpholin-4-yl)ethan-1-amine di-acetate; 2-(2-methylmorpholin-4-yl)ethan-1-amine di-sulfate: 1-(2-methoxyethyl)pyrrolidin-3-amine di-chloride; 1-(2-methoxyethyl)pyrrolidin-3-amine di-bromide; 1-(2-methoxyethyl)pyrrolidin-3-amine di-acetate; 1-(2-methoxyethyl)pyrrolidin-3-amine di-sulfate; 4-amino-3-[dimethylamino)methyl]butan-2-ol di-chloride; 4-amino-3-[(dimethylamino)methyl]butan-2-ol di-bromide; 4-amino-3-[(dimethylamino)methyl]butan-2-ol di-acetate; 4-amino-3-[(dimethylamino)methyl]butan-2-ol di-sulfate; 3-(morpholin-4-yl)propan-1-amine di-chloride; 3-(morpholin-4-yl)propan-1-amine di-bromide; 3-(morpholin-4-yl)propan-1-amine di-acetate; 3-(morpholin-4-yl)propan-1-amine di-sulfate; 3-(2-methylpiperazinyl-1-yl)propan-1-ol di-chloride: 3-(2-methylpiperaziny-1-yl)propan-1-ol di-bromide; 3-(2-methylpiperaziny-1-yl)propan-1-ol di-acetate; 3-(2-methylpiperziny-1-yl)propan-1-ol di-sulfate; 1-butyl pyridinium chloride; 1-butyl pyridinium bromide; 1-butyl pyridinium acetate; 1-butyl pyridinium methyl sulfate; 1-butyl-4-methyl pyridinium chloride; 1-butyl-4-methyl pyridinium bromide; 1-buty-4-methyl pyridinium acetate; 1-butyl-4-methyl pyridinium methyl sulfate; 1-butyl-3-methyl pyridinium chloride; 1-butyl-3-methyl pyridinium bromide; 1-butyl-3-methyl pyridinium acetate; 1-butyl-3-methyl pyridinium methyl sulfate; N-ethyl-N-methyl pyrrolidium chloride; N-ethyl-N-methyl pyrrolidium bromide; N-ethyl-N-methyl pyrrolidium hexafluorophosphate; N-ethyl-N-methyl pyrrolidium BF₄; N-ethyl-N-methyl pyrrolidium acetate; N-ethyl-N-methyl pyrrolidium methyl sulfate; N-butyl-N-ethyl pyrrolidium chloride; N-butyl-N-methyl pyrrolidium bromide; N-buty-N-methyl pyrrolidium acetate; N-butyl-N-methyl pyrrolidium methyl sulfate; N-butyl-N-methyl pyrrolidium iodide; N-butyl-N-methyl pyrrolidium BF₄; N-butyl-N-methyl pyrrolidium hexafluorophosphate; Ferric ammonium citrate; Ferrous ascorbate; Calcium L-ascorbate; Biotin; Butylated hydroxyanisole; Triethyl citrate; and any combination thereof.

9. The oral care composition according to claim 1 wherein the compound is selected from one or more of (2-chloro-3-hydroxypropyl)trimethylammonium chloride; (2,3-dihydroxypropyl)trimethylammonium chloride; 1-butyl pyridinium bromide; 1-butyl-4-methyl pyridinium chloride;
1-butyl-4-methyl pyridinium bromide; 1-butyl-4-methyl pyridinium iodide;
1-butyl-4-methyl pyridinium BF₄; 1-butyl 4-methyl pyridinium hexafluorophosphate; N-buty-N-ethyl pyrrolidium hexafluorophosphate;
N-butyl-N-ethyl pyrrolidium BF₄ N-butyl-N-methyl pyrrolidium chloride;
N-butyl-N-methyl pyrrolidium bromide; N-butyl-N-methyl pyrrolidium iodide;
N-butyl-N-methyl pyrrolidium BF₄; N-butyl-N-methyl pyrrolidium hexafluorophosphate; and any combination thereof.

10. The oral care composition according to claim 1 wherein the compound is choline bitartrate.

11. The oral care composition according to claim 1 wherein the composition is for removing or reducing plaque.

12. The oral care composition according to claim 1 wherein the composition is for inhibiting growth of bacteria.

13. The oral care composition according to claim 1 wherein the composition is for teeth whitening.

14. The oral care composition according to claim 1 wherein the composition is for preventing or treating a disease or condition of the oral cavity.

15. A method of removing or reducing plaque from the oral cavity of a subject comprising administering a therapeutically effective amount of a composition according to claim 1 to the subject.

16. A method of whitening teeth in a subject comprising administering a therapeutically elective amount of a composition according to claim 1 to the subject.

17. A method of reducing or inhibiting biofilm formation in the oral cavity of a subject comprising administering a therapeutically effective amount of a composition according to claim 1 to the subject.

18. A method of reducing the amount of bacteria in the oral cavity of a subject comprising administering a therapeutically effective amount of a composition according to claim 1 to the subject.

19-21. (canceled)
